(19) United States
(12) Patent Application Publication
(10) Pub. No.: US 2014/0158133 A1
(43) Pub. Date: Jun. 12, 2014

(54) Nasal Venti System

(71) Applicant: Olivia Frances Acosta, Berwyn, IL (US)

(72) Inventor: Olivia Frances Acosta, Berwyn, IL (US)

(21) Appl. No.: 13/706,520

(22) Filed: Dec. 6, 2012

(51) Int. Cl. A61M 16/06; A61M 16/00

(52) U.S. Cl. CPC A61M 16/0666; A61M 16/0057
USPC 128/204.25; 128/205.25

(57) ABSTRACT

A portable nasal ventilation assembly and system having a nasal piece with fluid delivery ports which engage and occlude the nares of a patient and provides sustained oxygen saturations greater than 90% with lung afflictions requiring high-flows such as Pulmonary Fibrosis. The assembly is noninvasive and does not require the use of positive pressure machines that can only be used within the confines of a hospital intensive care unit. It can be used on patients with different respiratory problems. Patients on the system can be home while awaiting further hospital treatment, such as (for example) a lung transplant, for a fraction of the cost of a hospital stay. Although the system does not replace ventilator devices, it is effective for spontaneously breathing patients that require ventilators to maintain an adequate life sustaining oxygen level. The device allows for portability and can reduce hospital stay and cost dramatically.

### TECHNICAL FIELD

The present device relates to ventilation systems. More specifically, the device relates to non-invasive, nasal ventilation systems.

### BACKGROUND OF THE INVENTION

Breathing is an involuntary action that most people probably take for granted. We breathe to get oxygen into our lungs and ultimately to oxygenate blood so that the oxygen can be carried to all parts of the body where it is needed. However, struggling to breath, even for a moment, is an experience which can cause panic in the strongest of individuals. Every day patients with most any type of chronic respiratory disorder can know the panic of struggling to take a normal breath, and then another. Many of such patients are admitted to a hospital where they can be connected to a mechanical assisted breathing machine (i.e., a ventilator) that alternately pushes air (possibly including a high-concentration of oxygen) into the lungs and withdraws carbon dioxide from the lungs, either invasively or noninvasively.

In the extreme cases, when admitted to a hospital, a breathing tube is inserted into the oral airway or trachea of a respiratory patient suffering from exacerbation of a pulmonary disease or a chronic respiratory disorder. The air tube, when connected to a mechanical ventilator, assists the patient's breathing by delivering air directly into the lungs. This regimen is usually maintained until the patient can sustain oxygen levels and be either weaned-off mechanical ventilation entirely or sometimes placed on a noninvasive bi-level positive airway pressure system.

If the oxygenation issues cannot be resolved, patients can become chronically dependent on the stationary mechanical breathing machines. Prolonged use of the invasive ventilator system increases the risk of pneumonia.

In an effort to reduce the need for invasive and non-invasive mechanical ventilation, a portable noninvasive nasal ventilation system for spontaneously breathing patients is disclosed. The system can aide in the treatment of some respiratory disorders without the use of a breathing machine.

### SUMMARY OF THE INVENTION

There is disclosed herein a noninvasive ventilation assembly and portable ventilation system which avoid the disadvantages of prior devices while affording additional structural and operating advantages.

Generally speaking, the portable noninvasive fluid delivery system comprises a portable fluid source, a fluid delivery tube having a first end for connecting to the fluid source and a second end opposite the first end, and at least one fluid discharge port configured to engage and occlude a nasal passage of a user. Preferably, there are two fluid discharge ports configured to engage and occlude both nasal passages of a user.

In an embodiment of the assembly, the fluid delivery tube comprises two flexible tubes connected to a Y-connector and a flow control device positioned either along the delivery tube between the first end and the second end or coupled to the portable fluid source. Preferably, a venturi device within the fluid delivery tube mixes air with a delivery fluid, such as oxygen.

In a most preferred embodiment of the noninvasive breathing assembly, the assembly comprises two soft corrugated tubes, a pressure monitoring port and cap connected to one end of each tubing and including a soft nosepiece having ports configured to enter and fit flush against a user's nasal openings, a Y-shaped coupling connected to the tubes opposite the nosepiece, a length of tubing connected to a base opening of the Y-shaped coupling, a fluid entrainment device to control flow levels, and a length of tubing connected at one end to the entrainment device and at another end to a fluid source. The fluid entrainment device is preferably a venturi which creates a greater flow and a positive pressure within the lungs (PEEP).

These and other aspects of the invention may be understood more readily from the following description and the appended drawings.

### BRIEF DESCRIPTION OF THE DRAWINGS

For the purpose of facilitating an understanding of the subject matter sought to be protected, there are illustrated in the accompanying drawings, embodiments thereof, from an inspection of which, when considered in connection with the following description, the subject matter sought to be protected, its construction and operation, and many of its advantages should be readily understood and appreciated.

FIG. 1 is a perspective view of an embodiment of the present invention;

FIG. 2 is an exploded view of the embodiment of FIG. 1;

FIG. 3 illustrates the use of an embodiment of the present invention; and

FIG. 4 is a top view of the embodiment of FIG. 1.

### DETAILED DESCRIPTION OF THE INVENTION

While this invention is susceptible of embodiments in many different forms, there is shown in the drawings and will herein be described in detail at least one preferred embodiment of the invention with the understanding that the present disclosure is to be considered as an exemplification of the principles of the invention and is not intended to limit the broad aspect of the invention to any of the specific embodiments illustrated.

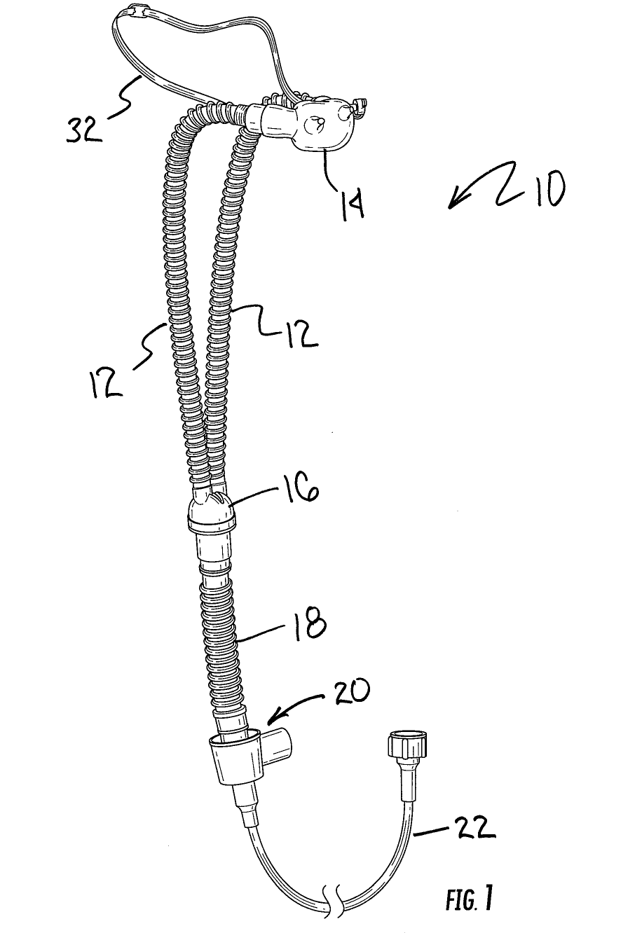

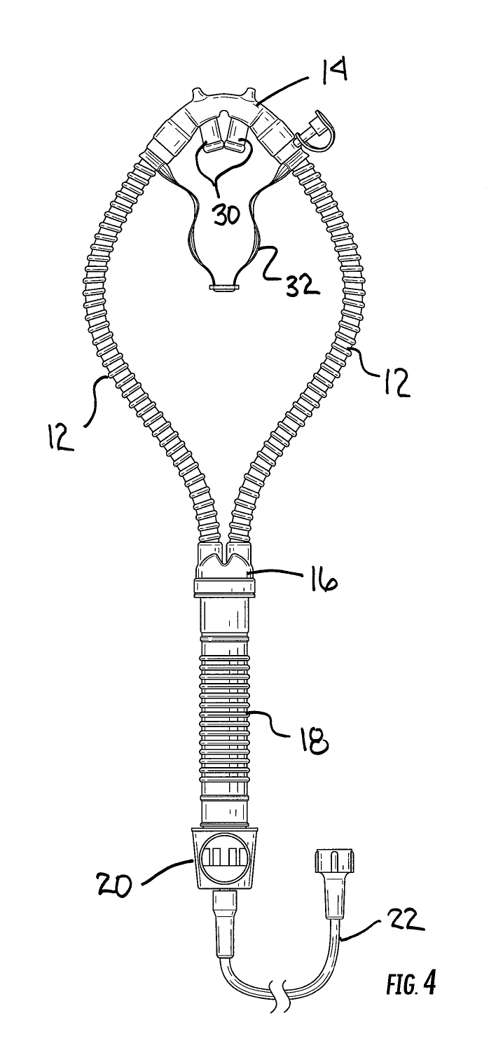

Referring to FIGS. 1-4, there is illustrated a noninvasive breathing assembly, generally designated by the numeral 10. The particular illustrated assembly 10 is for use with a portable fluid source, such as an oxygen tank. However, it should be understood that the principles of the invention may be more broadly applied to other analogous uses and devices.

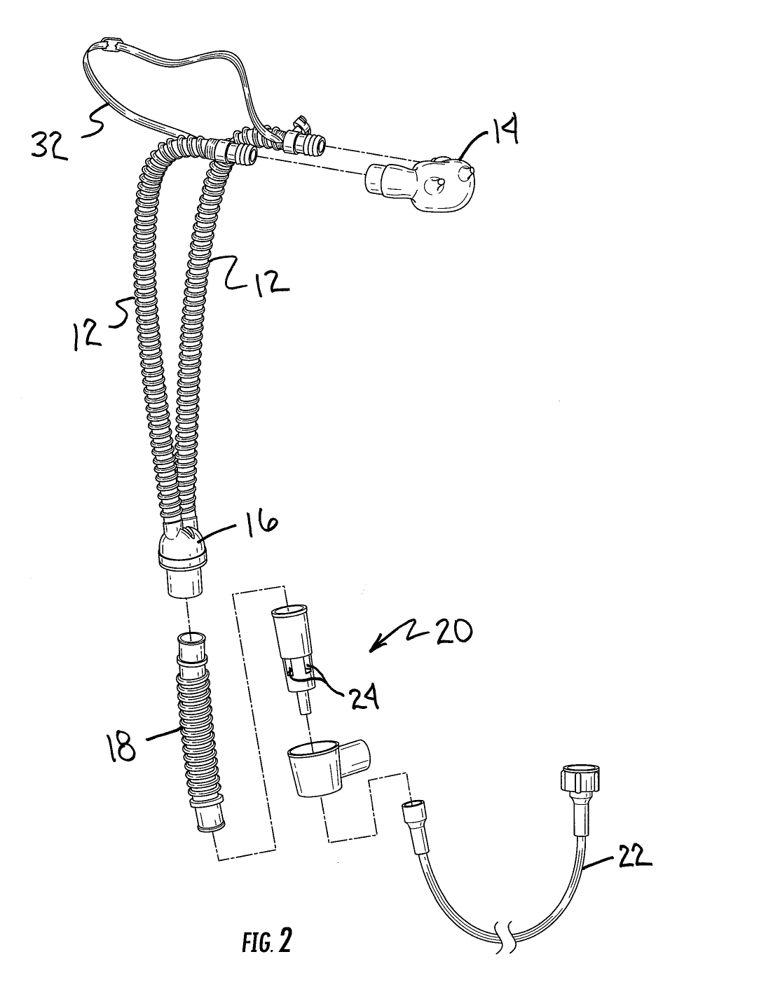

As can be seen in FIGS. 1 and 2, the assembly 10 is comprised of parts that are commonly used to deliver high-flow noninvasively. Specifically, the assembly 10 comprises a pair of corrugated flexible tubes 12 connected by a first end to a nasal piece 14, and by a second end to a Y-shaped connector 16. The Y-shaped connector 16 then connects to another corrugated flexible tube 18 (preferably of a larger diameter than the tubes 12), which couples to a venturi device 20. Finally, the venturi device 20 is connected to a length of standard gas delivery tubing 22 which secures to a fluid source (not shown).

The assembly 10 requires a fluid or flow source of a medical gas (not shown) connected to the venturi device 20 via standard oxygen tubing 22. The combination of the gas flow and the venturi device 20, which pulls in air at openings 24, creates a high-flow for delivery to a patient.

The venturi (or air-entrainment) device 20 is preferably selected from one of the several devices sold with color-coding to indicate oxygen concentration delivered—e.g., 24%, 26%, 28%, 30%, 35%, 40% and 50%. Regardless of the oxygen flow, the venturi device 20 entrains the same ratio of air to oxygen to maintain delivery of the rated oxygen concentration. To adjust the concentration of oxygen, one venturi device can be easily swapped out for another desired venturi device. These venturis are well-known devices in the industry and are commonly used by medical care personnel.

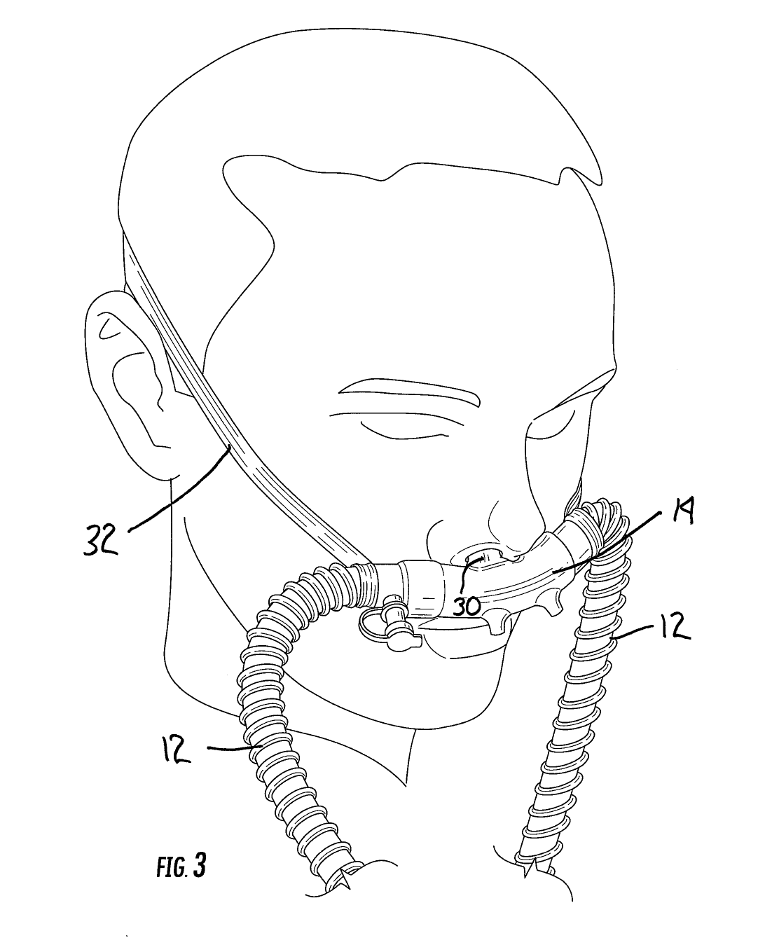

The soft nasal piece 14 includes two fluid delivery ports 30 which extend sufficiently from the nasal piece 14 to be inserted well into the nasal passage (or nares) of a user, as shown in FIG. 3. Further, the base of the ports 30 serve to substantially if not completely occlude the nares of the user, which insures that fluid being delivered is directed into the airway without escaping around the ports. The result is a positive air pressure (PEEP) in the user's lungs.

A expandable head strap 32 may be used to hold the nasal piece 14 in place. The head strap 32 should be adjustable to provide comfort to the user, as the device may be worn for a prolonged period.

In use, the disclosed portable system can be used on patients previously unable to leave the intensive care unit because they are connected to a ventilator. When connected to the portable system, patients are able to be transitioned from intensive care to a general hospital unit and eventually released to go home on the system.

The high-flows that are capable of being generated by the assembly 10 when connected to a flow source can decrease fluid within the lungs in cases of pulmonary edema, until the fluid is removed with medication. Further, the high-flows can be used to improve oxygenation for patients with afflictions such as Pulmonary Fibrosis and other chronic lung diseases.

The disclosed portable ventilation system is not intended to take the place of a mechanical ventilator. However, the system has been found to facilitate oxygenation of some spontaneously breathing patients with restrictive lung problems as a means to avoid the necessity of placing them on a mechanical ventilator.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

What is claimed is:

1. A portable noninvasive fluid delivery system comprising:
a portable fluid source;
a fluid delivery tube having a first end for connecting to the fluid source and a second end opposite the first end; and
at least one fluid discharge port configured to engage and occlude a nasal passage of a user.

2. The noninvasive breathing system of claim 2, wherein there are two fluid discharge ports configured to engage and occlude both nasal passages of a user.

3. The noninvasive breathing system of claim 1, wherein the fluid delivery tube comprises two flexible tubes connected to a Y-connector.

4. The noninvasive breathing system of claim 1, further comprising a flow control device positioned either along the delivery tube between the first end and the second end or coupled to the portable fluid source.

5. The noninvasive breathing system of claim 3, further comprising a venturi device within the fluid delivery tube for mixing air with a delivery fluid.

7. The noninvasive breathing system of claim 2, wherein the at least one fluid discharge port is comprised of a soft flexible material.

8. The noninvasive breathing system of claim 3, wherein the two discharge ports are comprised of a soft flexible material.

9. The noninvasive breathing system of claim 8, wherein the two discharge ports are configured to extend into a user's nasal passage.

10. The noninvasive breathing system of claim 9, further comprising a head strap for securing the at least one fluid discharge port within a user's nasal passage.

11. A noninvasive breathing assembly comprising:
two soft corrugated tubes;
a pressure monitoring port and cap connected to one end of each tubing and including a soft nosepiece having ports configured to enter and fit flush against a user's nasal openings;
a Y-shaped coupling connected to the tubes opposite the nosepiece;
a length of tubing connected to a base opening of the Y-shaped coupling;
a fluid entrainment device to control flow levels; and
a length of tubing connected at one end to the entrainment device and at another end to a fluid source.

12. The noninvasive breathing assembly of claim 11, wherein the oxygen entrainment device comprises a venturi adapter to control oxygen and flow levels.

13. The noninvasive breathing assembly of claim 1, wherein the venturi creates a greater flow and a positive pressure within the lungs (PEEP).
